(19) United States
(12) Patent Application Publication
(10) Pub. No.: US 2003/0059445 A1
(43) Pub. Date: Mar. 27, 2003

(54) Process for cultivating mites, nutrient preparation for this process, and preparation of allergenic extracts from these mites

(76) Inventors: Claude Andre, Saint Cyr au Mont d'Or (FR); Thierry Batard, Versailles (FR)

Correspondence Address: BIERMAN MUSERLIAN AND LUCAS, 600 THIRD AVENUE, NEW YORK, NY, 10016

(21) Appl. No.: 10/084,395

(22) Filed: Feb. 25, 2002

Related U.S. Application Data
(60) Provisional application No. 60/281,165, filed on Apr. 3, 2001.

(30) Foreign Application Priority Data
Mar. 1, 2001 (FR) 0102835

(51) Int. Cl. A61K 39/35

(52) U.S. Cl. 424/275.1

(57) ABSTRACT

The invention relates to a medium for cultivating and producing mites, and especially mites belonging to at least one of the following species: Dematophagoides pteronyssinus, Dermatophagoides farinae, Blomia kulagini or tropicalis, Pyroglyphus africanus, and Euroglyphus maynei. This medium is free from human or animal elements or proteins and comprises, in effective amounts, a plurality of amino acids in particulate form with a particle size of less than 250 μm, or in lyophilised form.

The present invention relates to a process for cultivating mites or acari with a view to producing allergenic extracts from mites, and nutrient formulations intended to be used in these processes.

Different species of mites are used to prepare allergenic extracts used in allergy formulations to act, for example, as in vivo or in vitro allergy tests, or in desensitising preparations given to the patients.

These mites include, in particular, the following species: Dermatophagoides pteronyssinus, Dermatophagoides farinae, Blomia kulagini or tropicalis, Pyroglyphus africanus, and Euroglyphus maynei, which are housemites feeding mainly on human skin scales or squamae.

Conventionally, these mites are produced using culture media containing suitably treated human scales autoclaved to inactivate viruses, bacteria and unconventional transmissible agents such as prions. The mites thus produced make it possible to obtain, by extraction, high quality allergenic extracts which are virtually free from allergens of other origins capable of producing cross-reactions which may falsify the tests or give rise to allergies.

Other methods of cultivating mites are known, using nutrient media based on proteins such as shrimp eggs or powdered pig's liver. These media produce satisfactory yields but contain non-acarian substances, particularly of animal origin, which may cause allergies and/or have an infectious potential.

Although the methods of inactivation used to treat human skin scales intended for the cultivation of mites are extremely effective and contamination with conventional or unconventional infectious agents is highly improbable, it is nevertheless desirable to use nutrient media of non-human and non-animal origin and free from any elements which might be allergenic to cultivate the mites.

The present invention therefore sets out to provide a process for cultivating and producing mites, and especially mites of the species mentioned above, which minimises the risk of the presence of infectious agents of animal or human origin.

Another objective of the invention is to provide a process of this kind which results in a substantial yield of mites.

Yet another objective of the invention is to improve the antigenicity of the mites intended to be used or extracted to produce the final formulations.

Yet another objective of the invention is to enable a large proportion of the culture medium to be eliminated easily.

The invention relates to a process for cultivating and producing mites, and especially mites belonging to at least one of the following species: Dermatophagoides pteronyssinus, Dermatophagoides farinae, Blomia kulagini or tropicalis, Pyroglyphus africanus, and Euroglyphus maynei, characterised in that the mites are cultivated on a medium free from human or animal elements or proteins and comprising, in effective amounts, a plurality of amino acids in particulate form with a particle size of less than 250 μm, or in lyophilised form.

The desired particle size of amino acids may be obtained by grinding the amino acids, individually or in admixture.

In equivalent manner, the amino acids may be obtained by dissolving the amino acids, and then, lyophilising them. In this case it is preferable for the lyophilisation to produce particles smaller than 250 μm.

Of course, in the medium according to the invention, only some of the acids may have been ground and/or lyophilised, especially as a function of their physical characteristics, for example their solubility, and, if need be, some acids may be added to the mixture just as they are.

The invention is based on the discovery that if commercially available amino acids are used just as they are (without grinding and/or without solubilisation-lyophilisation and/or without the addition of salts), the mites grow extremely poorly and the yields are very low. Surprisingly, the mixtures of amino acids having the features defined in the invention result in yields that are comparable or even superior to the conventional yields using human skin scales.

The mixtures of amino acids preferably comprise the majority or all of the amino acids which naturally constitute proteins. By the majority is meant at least 50%, for example 60 to 80%, of the twenty amino acids which naturally make up proteins or equivalent assimilable amino acids. However, it is also possible to add amino acids which do not constitute proteins, or to replace some of the amino acids with amino acids which do not constitute proteins.

In one particular embodiment, a mixture of amino acids resembling the composition of keratin or the stratum corneum may advantageously be used.

However, in alternative embodiments of the invention, it is also possible to use mixtures of amino acids mimicking the composition of shrimp eggs or soya. Other formulations may move away from this distribution.

The respective proportions of the amino acids may resemble the quantitative proportions of the amino acids in substances such as keratin, or the stratum corneum, shrimp eggs or soya, but there is by no means any requirement that the proportional distribution should be identical, provided that the individual amino acids are present in sufficient amounts.

The nutrient medium which contains the mixture of amino acids in the process, according to the invention, may also contain other normal ingredients of nutrient media for mites, intended either to provide a nutritional supplement or to give the medium a texture suitable for the development and multiplication of the mites, as well as salts.

Thus, it is preferably to incorporate wheat germs and/or yeast, especially baker's yeast, and/or cyanocobalamine and/or d-biotin, in the culture medium. The medium may also contain other vitamins.

The wheat germs are preferably heated to eliminate any risk of an allergic reaction.

In the process according to the invention, the culture medium, containing the mixture of amino acids, is brought to a suitable level of humidity which is normal for the mites being cultivated, and is kept at the appropriate usual temperature. The cultivation times may be three months, for example, while conventional periods of 2 to 5 months are preferred.

The invention also relates to the culture media containing the mixtures of amino acids according to the invention.

The invention also relates to the processes for preparing extracts or formulations of allergens obtained from the mites cultivated by the process according to the invention.

Further features and advantages of the invention will become apparent from reading the following description which is provided as a non-restrictive example.

Cultures were set up simultaneously on amino acid media according to Examples 1 and 3 and on human skin scales according to Example 2.

### EXAMPLE 1

This example describes a process of cultivation in a medium containing a commercial amino acid preparation.

A culture medium is prepared containing wheat germs, cyanocobalamine, baker's yeast and D-biotin.

The wheat germs are autoclaved at 121° C. for 20 min. then ground and screened through a 250 μm mesh.

The cyanocobalamine is ground and screened through a 250 μm mesh.

The baker's yeast is heated to 122° C. for 2 to 3 min., then to between 100 and 135° C. for 12 sec. in a drum rotating at a speed of 5 rpm, then heated to 100° C. for 15 min.

The commercial amino acid preparation was obtained from the company Frésénius-Kabi France S.A. It has the following composition, qs for 1 L of water PPI:

| | | |
|---|---|---|
| L-alanine | 3.8 | g |
| L-arginine | 4.2 | g |
| L-aspartic acid | 5.2 | g |
| L-cysteine hydrochloride monohydrate | 1.7 | g |
| expressed as L-cysteine/L-cystine | | |
| L-glutamic acid | 11.5 | g |
| glycine | 2.7 | g |
| L-histidine | 3.1 | g |
| L-isoleucine | 5.0 | g |
| L-leucine | 6.7 | g |
| L-lysine hydrochloride, expressed as L-lysine | 5.0 | g |
| L-methionine | 2.4 | g |
| L-phenylalanine | 7.0 | g |
| L-proline | 10.3 | g |
| L-serine | 9.6 | g |
| L-threonine | 3.8 | g |
| L-tryptophane | 1.3 | g |
| L-tyrosine | 0.6 | g |
| L-valine | 5.5 | g |
| calcium chloride dihydrate | 0.44 | g |
| magnesium sulphate heptahydrate | 0.493 | g |
| sodium hydroxide | 2.6 | g |
| potassium hydroxide | 0.70 | g |
| potassium chloride | 0.078 | g |

The solution is lyophilised and the lyophilisate collected is screened through a vibrating screen with a pore size of 250 μm.

The medium itself is prepared as follows:

For 600 g of medium:

252 g of screened wheat germs, 252 g of screened yeasts, 90 g of the lyophilised and screened amino acid solution, 5.4 g of screened cyanocobalamine and 0.6 g of D-biotin are weighed, the whole is homogenised in a homogeniser and then passed through a screen with a mesh size of 400 μm. The medium is packaged in labelled stoppered bottles and can be kept in a cold room at temperatures between +2° C. and +8° C.

The culture itself is carried out as follows:

The prepared media are seeded with a sample of conventional seed cultures of Dermatophagoides pteronisynus. Cultivation takes place in bottles at a temperature of 25° C., at a humidity level of 75%. The samples are taken at different times. The media harvested are then lyophilised and extracted at 5% in a 4 g/l ammonium bicarbonate solution for 24 hours at +4° C., then centrifuged at 3000 rpm for 15 minutes at +4° C. The supernatant is harvested and then filtered on a Millex HV filter with a pore size of 0.45 μm (Millipore).

In the extracts obtained the total allergenic activity is titrated (by Rast inhibition), the proteins are titrated (by the Lowry technique and/or the Bradford technique), and the major allergens Der p 1 and Der p 2 are titrated (using conventional titration kits).

The results of the Rast inhibition are expressed as RI/ml (RI: reactivity index), the extracts being titrated in comparison with a reference extract the activity of which is 100 RI/ml.

### EXAMPLE 2

The medium of Example 2 is identical to that of Example 1 except that the lyophilised commercial amino acid solution is replaced by a preparation of human skin scales.

These scales are autoclaved in bags at 134° C. for 18 min. The scales are then placed in stainless steel dishes and put into a drier at 37° C. for 1 to 2 weeks. The scales are then ground up and screened through meshes of 500 and 250 μm.

The powdered scales obtained are then treated with acetone and left to decant for 24 hours. The supernatant is eliminated. Then a final washing operation is carried out using acetone. The residue is collected and spread in thin layers over dishes and covered with a perforated sheet of aluminium. This substance is dried under a cover and finally screened through a vibrating screen with a mesh size of 250 μm.

Instead of the 90 g of amino acid mixture, the culture medium in this example comprises 90 g of the human skin scale preparation mentioned above.

### EXAMPLE 3

The culture medium contains wheat germs, cyanocobalamine and yeast as in Examples 1 and 2.

A mixture of amino acids is prepared as follows:

10 liters of sterile distilled water are poured into a container and the following amino acids are dissolved therein:

| | | |
|---|---|---|
| L-alanine | 17.2 | g |
| L-arginine | 26.4 | g |
| L-cysteine hydrochloride monohydrate | 4.4 | g |
| glycine | 49.6 | g |
| L-histidine | 5.2 | g |
| L-isoleucine | 13.2 | g |
| L-lysine hydrochloride | 20.0 | g |
| L-methionine | 8.0 | g |
| L-proline | 8.8 | g |
| L-serine | 44.0 | g |
| L-threonine | 13.6 | g |
| L-valine | 13.6 | g |
| L-tryptophane | 1.3 | g |
| L-phenylalanine | 20.8 | g |
| L-leucine | 34.8 | g |
| L-glutamic acid | 64.4 | g |
| L-aspartic acid | 9.7 | g |

The solution is then lyophilised and the lyophilisate collected is screened through a vibrating screen with a pore size of 250 μm.

6.5 g of aspartic acid and 4.0 g of tyrosine are ground up separately.

To prepare the medium, 79.5 g of the abovementioned lyophilised mixture, 6.5 g of ground aspartic acid and 4.0 g of ground tyrosine are added to the 252 g of wheat germs, 252 g of yeast, 5.4 g of cyanocobalamine and 0.6 g of D-biotin.

The mixture is homogenised and then screened through a 400 μm mesh. After seeding, cultivation is carried out as in Examples 1 and 2.

The comparative results of Examples 1 to 3 are shown in Table 1 for a first cultivation cycle, after 3 months, and in Tables 2 and 3 for a second cultivation cycle (the mites cultivated on a given medium are re-seeded onto the same medium), after 2.5 months and 3 months, respectively:

TABLE 1
Total allergenic activity, protein levels, of Der p 1 and Der
p 2 in 1/20 th extracts of Dermatophagoides pteronyssinus
cultivated on different media after three months' cultivation.

| | Amino acids | Human squamae | Amino acids |
|---|---|---|---|
| Medium containing | (example 1) | (example 2) | (example 3) |
| Total allergenic | 263 | 284 | 573 |
| activity (RI/ml) | | | |
| Protein level (μg/ml; | 537 | 544 | 217 |
| Bradford technique) | | | |
| Protein level (μg/ml; | 5689 | 6446 | 5538 |
| Lowry technique) | | | |
| Der p 1 (μg/ml) | 187.5 | 231.5 | 69.0 |
| Der p 2 (μg/ml) | 2.0 | 2.5 | 10.0 |

TABLE 2
Total allergenic activity, protein levels, of Der p 1 and Der
p 2 in 1/20 th extracts of Dermatophagoides pteronyssinus
cultivated for a second cycle on different media after two and
a half months' cultivation.

| | Amino acids | Human squamae | Amino acids |
|---|---|---|---|
| Medium containing | (example 1) | (example 2) | (example 3) |
| Total allergenic | 617 | 713 | 195 |
| activity (RI/ml) | | | |
| Protein level (μg/ml; | 328 | 371 | 75 |
| Bradford technique) | | | |
| Der p 1 (μg/ml) | 83.0 | 138.0 | 8.5 |
| Der p 2 (μg/ml) | 19.5 | 19.5 | 3.0 |

TABLE 3
Total allergenic activity, protein levels, of Der p 1 and Der
p 2 in 1/20 th extracts of Dermatophagoides pteronyssinus
cultivated for a second cycle on different media after three
months' cultivation.

| | Amino acids | Human squamae | Amino acids |
|---|---|---|---|
| Medium containing | (example 1) | (example 2) | (example 3) |
| Total allergenic | 631 | 486 | 192 |
| activity (RI/ml) | | | |
| Protein level (μg/ml; | 363 | 411 | 110 |
| Bradford technique) | | | |
| Der p 1 (μg/ml) | 206.0 | 267.5 | 23.0 |
| Der p 2 (μg/ml) | 14.0 | 4.5 | 5.0 |

It will be seen that, although cultivation on amino acids according to Example 3 gives good results during a first cultivation cycle, it gives poor results in a second cultivation cycle. This Example is therefore not suitable for routine cultivation of mites.

By contrast, cultivation on amino acids according to Example 1 seems to be highly suitable, not only because the results are satisfactory after two cultivation cycles, but because they are very similar to the results obtained with human squamae, which constitute the natural food of the mites under discussion here. The relative weakness of the results obtained in the first test can certainly be put down to an excessively long cultivation period (harvesting at two and a half months would certainly have given better results).

1. Medium for cultivating and producing mites, and especially mites belonging to at least one of the following species: Dermatophagoides pteronyssinus, Dermatophagoides farinae, Blomia kulagini or tropicalis, Pyroglyphus africanus, and Euroglyphus maynei, characterised in that it is free from human or animal elements or proteins and comprises, in effective amounts, a plurality of amino acids in particulate form with a particle size of less than 250 μm, or in lyophilised form.

2. Medium according to claim 1, characterised in that it comprises amino acids obtained by grinding amino acids.

3. Medium according to claim 2, characterised in that it also comprises lyophilised amino acids and/or amino acids obtained commercially.

4. Medium according to claim 1, characterised in that it comprises lyophilised amino acids and amino acids obtained commercially.

5. Medium according to claim 1, characterised in that it contains salts.

6. Medium according to claim 1, characterised in that the mixture of amino acids contains at least 50% of amino acids which naturally make up proteins or the equivalents thereof.

7. Medium according to claim 1 characterised in that the mixture of amino acids reproduces the spectrum of amino acids that make up keratin or the stratum corneum.

8. Medium according to claim 1, characterised in that the mixture of amino acids reproduces the spectrum of amino acids present in shrimp eggs or in soya.

9. Medium according to claim 7, characterised in that the respective proportions of the amino acids are similar to the quantitative proportions of the amino acids and salts in substances such as keratin or the stratum corneum or shrimp eggs or soya.

10. Medium according to claim 8, characterised in that the respective proportions of the amino acids are similar to the quantitative proportions of the amino acids and salts in substances such as keratin or the stratum corneum or shrimp eggs or soya.

11. Medium according to claim 1, characterised in that it also contains other conventional elements of nutrient media for mites, intended to provide supplementary nutrition and/or give the medium a texture suitable for the development and multiplication of the mites.

12. Medium according to claim 11, characterised in that it also contains wheat germs and/or yeast, especially baker's yeast, and/or cyanocobalamine and/or d-biotin.

13. Medium according to claim 1, characterised in that it may contain soya.

14. Medium according to claim 1, characterised in that it contains at least 50% of the following amino acids:
L-alanine
L-arginine
L-aspartic acid
L-cysteine/cystine
L-glutamic acid
glycine
L-histidine
L-isoleucine
L-leucine
L-Lysine
L-methionine
L-phenylalanine
L-proline
L-serine
L-threonine
L-tryptophane
L-tyrosine
L-valine

15. Medium according to claim 14, characterised in that it contains the amino acids in the following proportions, adding up to a total of 93.711 g:

| | | |
|---|---|---|
| L-alanine | 3.8 | g |
| L-arginine | 4.2 | g |
| L-aspartic acid | 5.2 | g |
| L-cysteine hydrochloride monohydrate | 1.7 | g |
| expressed as L-cysteine/L-cystine | | |
| L-glutamic acid | 11.5 | g |
| glycine | 2.7 | g |
| L-histidine | 3.1 | g |
| L-isoleucine | 5.0 | g |
| L-leucine | 6.7 | g |
| L-lysine hydrochloride, expressed as L-lysine | 5.0 | g |
| L-methionine | 2.4 | g |
| L-phenylalanine | 7.0 | g |
| L-proline | 10.3 | g |
| L-serine | 9.6 | g |
| L-threonine | 3.8 | g |
| L-tryptophane | 1.3 | g |
| L-tyrosine | 0.6 | g |
| L-valine | 5.5 | g |
| calcium chloride dihydrate | 0.44 | g |
| magnesium sulphate heptahydrate | 0.493 | g |
| sodium hydroxide | 2.6 | g |
| potassium hydroxide | 0.70 | g |
| potassium chloride | 0.078 | g |

- potassium hydroxide 0.70 g - potassium chloride 0.078 g

16. Process for cultivating and producing mites, and especially mites belonging to at least one of the following species: Dermatophagoides pteronyssinus, Dermatophagoides farinae, Blomia kulagini or tropicalis, Pyroglyphus africanus, and Euroglyphus maynei, characterised in that the mites are cultivated on a medium according to claim 1.

17. Process according to claim 16, characterised in that the culture medium, containing the mixture of amino acids, is brought to a suitable level of humidity, normal for the mites being cultivated, and maintained at the appropriate usual temperature.

18. Process according to claim 18, characterised in that cultivation is carried out for between 2 and 5 months.

19. Process for obtaining an allergenic preparation, characterised in that the allergens are extracted from the culture according to claim 16.
